(19) United States
(12) Patent Application Publication
(10) Pub. No.: US 2017/0007513 A1
(43) Pub. Date: Jan. 12, 2017

(54) ENCAPSULATED PERSONALIZING SUBSTANCE FOR IMPLANTATION IN THE SKIN

(71) Applicant: Chamber Works, LLC, Wilmington, DE (US)

(72) Inventors: Patrick Duffy, SR., Wilmington, DE (US); Patrick Duffy, JR., Wilmington, DE (US); Sherry Freebery, Wilmington, DE (US); Brian McVeigh, Kennett Square, PA (US); Edith Mathiowitz, Brookline, MA (US)

(21) Appl. No.: 15/206,971

(22) Filed: Jul. 11, 2016

Related U.S. Application Data
(60) Provisional application No. 62/190,624, filed on Jul. 9, 2015.

(51) Int. Cl. A61K 8/11; A61K 8/02; A61K 8/60; A61K 8/25; A61K 8/98; A61K 8/97; A61Q 1/00; A61K 8/19

(52) U.S. Cl. CPC A61K 8/11; A61Q 1/00; A61K 8/0279; A61K 8/19; A61K 8/25; A61K 8/981; A61K 8/97; A61K 8/606; A61K 2800/652; A61K 2800/651; A61K 2800/56; A61K 2800/412

(57) ABSTRACT

Compositions for delivering materials having personal significance to an individual are described herein. The compositions include particles encapsulating a personalizing substance, which can be any material of personal significance. The particles are made from a biocompatible, inert material, preferably a metal. In some embodiments, the composition includes the personalizing particles in a pharmaceutically acceptable carrier for delivery into the skin.

A method for delivering materials of personal significance to an individual are also described. The method includes administering particles containing personalizing substances to the recipient. The material is preferably delivered to the individual's skin.

Kits for obtaining a personalizing substance from an end user and kits for delivering the encapsulated personalizing substance to the end user are provided. The kits include equipment for obtaining a sample of the personalizing substance and a vial, or personalizing particles.

### CROSS REFERENCE TO RELATED APPLICATION

This application claims benefit of and priority to U.S. Provisional Patent Application No. 62/190,624 filed on Jul. 9, 2015, incorporated by reference in its entirety.

### FIELD OF THE INVENTION

The present invention relates to personalizing materials to be added to the skin.

### BACKGROUND OF THE INVENTION

Humans have been personalizing their skin for years, for example, by applying tattoos to the skin. The first recorded formula for mixing and applying tattoo ink dates back to the fifth century and is attributed to the Roman physician Aetius. Tattoo inks were derived from natural substances and comprised a suspension of pigmented particles in a liquid carrier. Applying tattoo ink with needles or similar instruments to the skin, where the ink remains permanently, produces tattoos. This technique introduces the pigment suspension through the skin by an alternating pressure-suction action caused by the elasticity of the skin in combination with the movement of the needle. Water and other carriers for the pigment introduced into the skin diffuse through the tissues and are absorbed. Once the skin has healed, most pigment particles remain in the interstitial space of the tissue.

Tattoos are used for a variety of reasons, primarily for ornamentation of the skin. See U.S. Pat. No. 6,013,122 to Klitzman & Koger. However, tattoos are routinely used to create a physical connection with loved ones, for example, by having a tattoo of their name placed on a recipient's body.

A personalized ink tattoo creates a physical connection with a person, object, place, or event, because the personalized tattoo incorporates into the tattoo ink and, therefore, the image displayed in the skin, a personalizing substance. There is still a need for compositions which can be used to personalize the skin using expressions other than words and pictures, for example, compositions which include material of personal significance to the subject, and which can remain inert over time, when delivered to the subject.

It is an object of the present invention to provide inert compositions which include materials of personal significance to a subject.

It is also an object of the present invention to provide methods of delivering materials of personal significance to a subject.

### SUMMARY OF THE INVENTION

Compositions for delivering materials having personal significance to an individual (referred to herein as a “personalizing substance”) are described herein. The compositions include particles encapsulating a personalizing substance, referred to herein as, “personalized capsules” or “personalized particles”. The particles are made from a biocompatible material, preferably a metal, most preferably titanium. The personalizing substance can be a biological material, sand, soil, metal, water, sea water, holy water, synthetic or biological polymers, cremated ash, ceramics, animal or plant tissue, or another physiologically compatible component having personal significance. A preferred particle is in the form of a capsule, which includes a cavity for placing the personalizing substance, and a seal, which can be permanent or removable.

A method for delivering materials of personal significance to an individual is also described. The method includes administering particles containing personalizing substances to the recipient. The material is preferably delivered to the individual's skin. Following delivery to an individual's skin, the encapsulated material remains in the particles and the particles do not erode. The encapsulated material remains in the particles/capsules for as long as it is in the individual's body, such as for at least 5 years, at least 10 years, at least 15 years, at least 20 years, or for a longer period of time.

Kits for obtaining a personalizing substance from an end user and kits for delivering the encapsulated personalizing substance to the end user are provided. The kit for obtaining personalizing substance includes equipment for obtaining a sample of the personalizing substance and a vial. The equipment may be tailored to the nature of the personalizing substance that will be provided. The kit for delivering the personalizing substance to the end user includes personalized particles/capsules, alone, or in a pharmaceutically acceptable carrier. In some embodiments, the kit includes a needle of appropriate size for injecting/implanting the personalized particle/capsule to a site in need thereof, for example, the skin.

### BRIEF DESCRIPTION OF THE DRAWINGS

FIG. 1A shows an exemplary titanium personalized capsule (10) which is cylindrical in shape, and having engravings (12). FIG. 1B shows a cross section (20) of the titanium capsule and exemplary personalizing substance (DNA microspheres (22)) in the capsule.

FIG. 2A shows an exemplary spherical personalized capsule (30), including optional groves (32a, 32b, etc.) etched along the circumference of the exterior of the capsule (30). FIG. 2B shows a cross section (32) of the spherical capsule (30) shown in FIG. 2A, and exemplary personalizing substance (DNA microspheres (22)) therein.

FIG. 3A shows an exemplary cylindrical capsule (40) with a base (42) and a cap (44). The base (400) includes the opening of a cavity (50) and threads (48) on the outer surface. The cap (44) includes threads (46), configured to mate with corresponding threads (48) on the capsule base (42). FIG. 3B shows a cross section (52) of the exterior of the device shown in FIG. 3A, showing the cap (56) in the locked position with the base (54) and exemplary personalizing substance (DNA microspheres (22)) therein. FIG. 3C shows an example of the cap (44) with an engraving (58).

FIG. 4A shows an exemplary cylindrical capsule (60), which includes a base (62) and a screw in cap (64), with the cap in the closed (screwed in) position. The cap (64) includes decorative grooves (66a, 66b and 66c). FIG. 4B shows an exemplary cylindrical capsule (70) with an etched base (72) and the cap (64) including one decorative groove (66). FIG. 4C shows an exemplary spherical personalized particle (80) with decorative engraving (82).

### DETAILED DESCRIPTION OF THE INVENTION

### I. Definitions

As used herein, the term “personalizing substance” refers to a material of significance to an individual. The personalizing substance may be a natural or synthetic material, where at least a portion of the material is capable of being encapsulated in a microparticle. The term personalizing substance is used herein to refer to the material both prior to and subsequent to encapsulation.

As used herein, the term “personalized capsule” refers to a particle, which can be in a variety of different shapes and sizes, containing one or more personalizing substances. The terms “personalized capsule” and “personalized particle” are used interchangeably herein.

As used herein, the term “biological material” means any biological substance, including, but not limited to biological micromolecules, such as a nucleotides, amino acids, cofactors, or hormones, biological macromolecules, such as nucleic acids, polypeptides, proteins (for example enzymes, receptors, secretory proteins, structural and signaling proteins, hormones, ligands, etc.), polysaccharides, and/or any combination thereof.

As used herein, the term “recipient” refers to the recipient of the encapsulated personalizing substance. The recipient may be any subject, human, animal or plant, capable of receiving the encapsulated personalizing substance.

As used herein the phrase “does not release the personalizing substance” refers to a personalized particle/capsule that does not release a substantial amount of the personalizing substance into a surrounding aqueous solution or buffer as detected by an in vitro assay as described herein.

As used herein, “nanoparticle” refers to a particle or a structure in the nanometer (nm) range, typically from about 1 to about 1000 nm in diameter.

As used herein, the term “small batch” refers to a batch size of an encapsulated personalizing substance suitable for use by no more than one, no more than two, no more than three, no more than four, no more than five, no more than six, no more than seven, no more than eight, no more than nine, or no more than ten individuals, optionally with a small amount remaining after application to the individual for verification purposes. In some embodiments, the batch size of the encapsulated personalizing substance is less than about 10,000, 5000, 4000, 3000, 2000, 1000, 500, 100, 50, 10, 1, 0.1 or 0.01 mg. Any of these values may be used to define a range for the batch size of the encapsulated personalizing substance. For example the batch size of the encapsulated personalizing substance may range from about 10,000 mg to about 0.01 mg, from about 10,000 mg to about 1000 mg, or from about 5000 mg to about 500 mg.

As used herein the term “vector” refers to a DNA molecule used in biotechnology for storage, propagation, delivery or integration of recombinant DNA. Examples of vectors include plasmid backbones, viral vectors, bacmids, cosmids, and artificial chromosomes.

### II. Compositions

Compositions for placing a personalizing substance in the skin of an individual, to remain at the site of placement, are described. The compositions include capsules/particles encapsulating a personalizing substance i.e., personalized capsules/particles. The capsules are preferably made from a biocompatible material, preferably a metal.

In some embodiments, the compositions are formulated for injection into skin. In this embodiment, the personalized capsule/particle is generally provided in a suitable pharmaceutically acceptable carrier for injection into the skin.

The compositions may be used to integrate substances of special significance to an individual into his/her skin.

A. Particles for Encapsulating Personalizing Substances

The concentration of a personalizing substance encapsulated in a capsule is presented as percent loading. Because values for the percent loading are dependent on the weights of the personalizing substances, percent loading values for the different personalizing substances may vary significantly. Therefore, different ranges for the percent loading for different personalizing substances are contemplated. In some embodiments, low concentrations (e.g., up to 0.1% w/w or lower) of the personalizing substance in the capsules are used.

In some embodiments, such as when the encapsulating material is isolated DNA, only a small sample is provided for encapsulation. In these embodiments, the particles typically contain low concentrations of DNA. However, if a large amount of the encapsulating material is provided, the loading of the encapsulating material in the personalized capsule can be higher as long as the resulting particles do not allow DNA to be released.

The particle can include about 0.00001, 0.00005, 0.0001, 0.0005, 0.001, 0.005, 0.01, 0.02, 0.03, 0.04, 0.05, 0.06, 0.07, 0.08, 0.09, 0.1, 0.5, 1, 2, 3, 4, 5, 6, 7, 8, 9 or 10% weight of the encapsulating material/weight of the particle (w/w). In some embodiments, the particles include less than about 0.00001, 0.00005, 0.0001, 0.0005, 0.001, 0.005, 0.01, 0.02, 0.03, 0.04, 0.05, 0.06, 0.07, 0.08, 0.09, 0.1, 0.5, 1, 2, 3, 4, 5, 6, 7, 8, 9 or 10% weight of the encapsulating material/weight of the particle (w/w). Any of these values may be used to define a range for the concentration of the encapsulating material in the particle. For example, the particles may contain encapsulating material in an amount ranging from about 0.00001 to about 10% w/w or from about 0.001 to about 2% w/w. In some embodiments, the amount of encapsulating material in the particles is less than about 0.1% w/w.

Typically, the percent loading of personalizing substances other than DNA is higher than the loadings of DNA in the particles. For example, the amount of the non-DNA personalizing substance in the particle may range from about 0.001 to about 10% w/w or from about 0.001 to about 2% w/w. Optionally, the amount of the non-DNA personalizing substance in the particle is less than about 0.001, 0.005, 0.01, 0.02, 0.03, 0.04, 0.05, 0.06, 0.07, 0.08, 0.09, 0.1, 0.5, 1, 2, 3, 4, 5, 6, 7, 8, 9 or 10% w/w. Any of these values may be used to define a range for the concentration of the substance in the particle. For example, the amount of the personalizing substance in the particle may range from about 0.001 to about 10% w/w or from about 0.001 to about 2% w/w.

1. Particle Type, Size and Shape

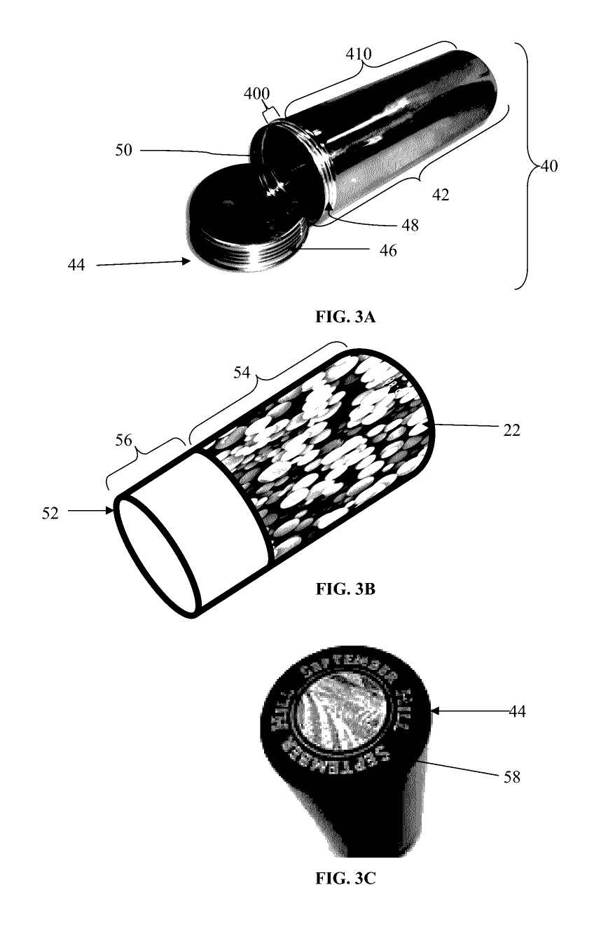

Particles that can be used to encapsulate personalizing substances include nanoparticles, microparticles, and centimeter-sized particles, collectively, capsules/particles. In some preferred embodiments, the personalizing particle is not micron or nano-sized. In these embodiments, the particle/capsule is millimeter or centimeter sized, and is preferably in the form of a hollow cylindrical tube. The capsule includes a cavity into which the personalizing substance is placed and a seal/cap, which can be permanent or removable. FIG. 3A shows an exemplary cylindrical capsule (40) with a base (42) and a cap (44). The base (400) includes the opening of a cavity (50) and threads (48) on an outer surface.

The size of the cavity can range from 0.5 mm to 10 mm in diameter for a circular device for example, or in height for a cylindrical device. Exemplary cavity sizes include 0.6 mm, 0.7 mm, 0.8 mm, 0.9 mm, 1mm, 2 mm, 3 mm, 4 mm, 5 mm, 6 mm, 7 mm, 8 mm, 9 mm and 10 mm. The size of the cavity can of course be varied to include values between the numbers disclosed herein, so long as the cavity size/capacity is large enough to contain the personalizing material to be included therein. The seal can be in the form of a screw on/in cap, or a snap on cap.

In some embodiments, the capsule is in the form of two pieces including base and a cap, which can be combined to form the capsule. For example, one piece (the base) is threaded at one end, and is configured to be received into one end of the second piece, which includes grooves configured to receive the threaded end of the first piece. In embodiments including a screw on cap, the cap includes an inner surface with thru cut threads configured to mate corresponding threads on the outer surface of one end of the capsule. The diameter of the cap in these embodiments is typically greater than the diameter of the end of the base on which the cap is screwed on.

FIG. 3A shows a cylindrical capsule (40) with a base (42) and a cap (44). The cap (44) includes threads (46), configured to mate with corresponding threads (48) on the capsule base (42). FIG. 3B shows an example of the cap (44) with engraving (58).

In embodiments including a cap that screws into a base, one end of the capsule is configured to receive a locking screw cap i.e., this end includes a cavity with an inner surface having threads configured to mate corresponding threads on a screw from the locking cap. The diameter of the cap in this embodiment is typically smaller that the diameter of the end of the base that contains the threads that mate with the corresponding threads on the cap.

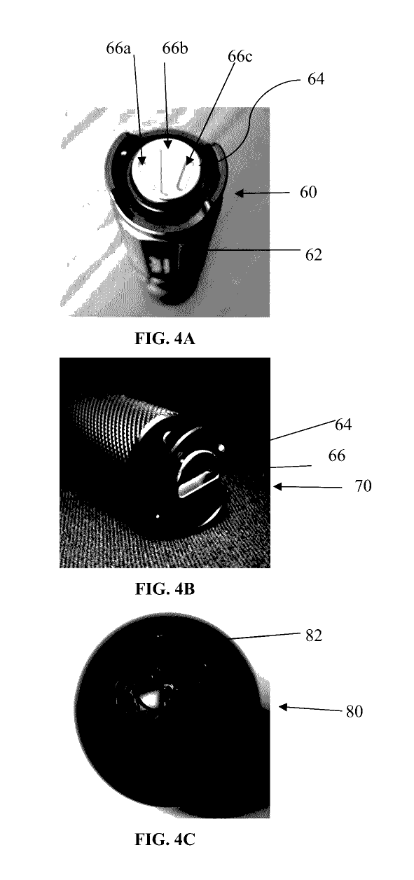

FIG. 4A is an exemplary cylindrical capsule (60), which includes a base (62) and a screw in cap (64), with the cap in the closed position. The base contains a cavity (not shown in Figure) configured to receive the personalizing substance. The base contains two substantially flat end portions; the upper end portion is visible in FIG. 4A. The base also contains an outer cylindrical wall. The cap (64) includes decorative grooves (66a, 66b and 66c). In this embodiment, the threaded cap fits into an interior recess on the upper portion of the base with corresponding threads that mate with the threads on the cap. The interior recess for receiving the cap is positioned on the upper end portion of the base is smaller in diameter than the outer diameter of the base.

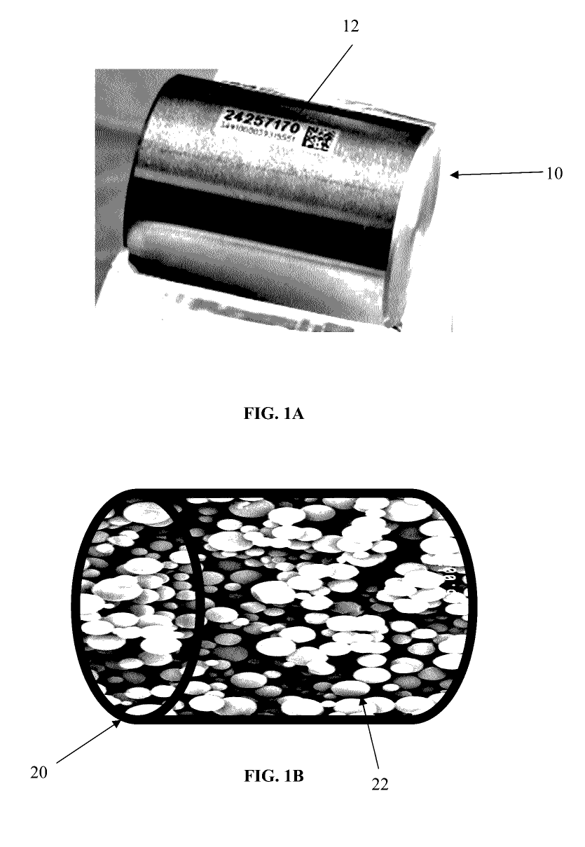

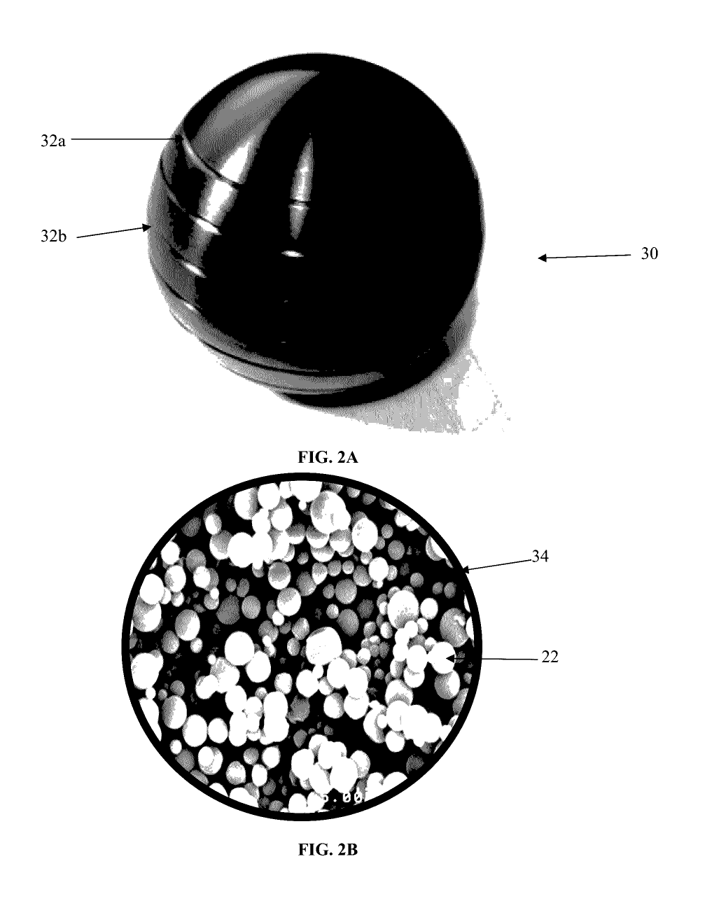

In some embodiments, the particle can have the size of a microchip. The particles can be made in any shape. For example, the particles can be cylindrical (e.g., FIGS. 1A, 4A, 4B), round, spherical (FIG. 2A), oval, oblong, in the shape of an urn or an animal etc. Other suitable shapes include, but are not limited to, flakes, triangles, rods, polygons, needles, tubes, cubes, and cuboid structures.

In some preferred embodiments, the particles can be administered with tattoo ink.

In other embodiments, the personalized particles can be administered using known devices for dermal implantation of particles of similar size.

2. Additional Personalization

The personalized particles can be further personalized, if desired, such as by engraving the exterior of the capsule. For example, the exterior of the particles can be engraved (for example using laser engraving) to include a picture of a loved one, image of a place, longitude and latitude coordinates, etc., or any sign/symbol of personal significance to the end user, with/without a connection to the personalizing substances within the particle.

An example of a word engraving is shown in FIG. 1A, which depicts a titanium capsule (10) which is cylindrical in shape, and having engravings (12).

Where the particle is a two piece particle i.e., including a base and a cap, the engraving can be on the base or the cap. FIG. 3C shows an example of the cap (44) with engraving (58). The engraving can be place on the particle as desired. FIG. 4C exemplifies a spherical personalized particle (80) with decorative engraving (82) on the exterior of the particle.

The personalized particle can be further personalized, if desired. For example, the cap can have one or more groves in parallel or angled. The base can be smooth or etched, as desired. For example, FIG. 4A shows a cylindrical capsule (60), which includes a base (62) and a screw in cap (64). The cap (64) includes decorative grooves (66a, 66b and 66c). FIG. 4B shows a cylindrical capsule (70) with an etched base (72), where the etching is on the outer surface of the cylindrical wall, and the cap (64) including one decorative groove (66).

a. Nano and Microparticles

The personalizing substance(s) can be encapsulated in nanoparticles or microparticles.

The diameter of the nanoparticle may be, for example, about 1000, 900, 800, 700, 600, 500, 400, 300, 200, 100, 90, 80, 70, 60, 50, 40, 30 or 20 nanometers (nm). In certain embodiments, the diameter of the nanoparticle is less than about 1000, 900, 800, 700, 600, 500, 400, 300, 200, 100, 90, 80, 70, 60, 50, 40, or 30 nanometers (nm). Any of these values may be used to define a range for the diameter of the nanoparticle. For example, the diameter of the nanoparticle may be from about 20 nm to about 1000 nm or from about 20 nm to about 100 nm.

In other embodiments, the personalizing substance is encapsulated in a microparticle. The core of the microparticles contains the personalizing substance, which is surrounded by a biocompatible matrix that forms the outer shell of the microparticle.

The microparticles can have a diameter of less than 10, 9, 8, 7, 6, 5, 4, 3, 2, 1, 0.9, 0.8, 0.7, 0.6, 0.5, 0.4, 0.3, 0.2 or 0.1 micron(s). Any of these values may be used to define a range for the diameter of the microparticle. For example the diameter of the microparticle may be from about 0.1 to about 10 microns, from about 0.1 to about 1 micron, or from about 0.1 to about 2 microns. Typically, the microparticle diameter is less than 5 microns. Preferably for compositions that are used as additives in a tattoo ink, the microparticle diameter ranges from about 1 to about 10 microns, more preferably from about 1 to 2 microns. Microparticles with a diameter of 10 microns and less may be introduced into the skin via a tattoo gun or any similar device. In other embodiments, larger microparticles or particles may be used. For example the microparticles may have a diameter of ranging from 10 microns to 1000 microns.

b. Larger Sized Particles/Capsules

The particles can be millimeter- or centimeter-sized in diameter, for example, the size of a microchip. For example, the capsules can have a size ranging from 0.1 cm to 3 cm, for example, the particle can be in a size of 0.1 -10 mm or from 10 mm to 3 cm. The cavity within the particle is selected to be smaller than the total size of the particle (FIG. 3A). For example, a particle that is 1 cm in diameter or height preferably has a cavity that is less than 1 cm in diameter, for example, 0.6 mm, 0.7 mm, 0.8 mm, 0.9 mm, 1 mm, 2 mm, 3 mm, 4 mm, etc., but less than 1 cm.

The loading capacity of larger particles is greater than the capacity disclosed for micro/nanoparticles and can range from 1-100% v/v of the cavity within the capsule. (see, e.g. FIGS. 1A and 3B).

In optional embodiments, the personalizing substance may be encapsulated in a polymeric nanoparticle/microparticle, prior to encapsulation into larger sized capsules disclosed herein. For example, the personalizing substance may be encapsulated in a polymeric nanoparticle prior to encapsulation into a metallic microparticle, or millimeter- or centimeter-sized capsule or it may encapsulated into a polymeric microparticle prior to encapsulation into a millimeter- or centimeter-sized capsule. Useful polymers and methods for encapsulating personalizing substances into polymers are disclosed in PCT/US2015/010822, filed Jan. 9, 2015. In this embodiment, the nano and microparticle size and loading disclosed above is generally applicable. The number of particles loaded into the metallic microparticle or millimeter- or centimeter-sized particle varies according to capacity of the cavity and can readily be calculated by one of ordinary skill in the art, using the size of the nano- or micro-particles, the volume of the cavity within the selected capsule, and the desired loading capacity.

In the preferred embodiment, the personalizing substance is included in the capsules disclosed herein without prior encapsulation in polymeric nano- or micro-particles.

3. Personalizing Substance

Generally, the compositions described herein include a personalizing substance. Suitable personalizing substances include, but are not limited to, biological materials such as, for example, animal or plant tissue, sand, soil, or rock particles, or compounds extracted from sand, soil, or rock, metal, sea water, holy water, synthetic or natural polymers, cremated ash, ceramics, and other physiologically compatible components.

In some embodiments, the compositions may contain DNA without any additional personalizing substances. In other embodiments, the compositions include DNA and one or more additional personalizing substances. For example, the additional personalizing substances may be one or more samples from sand, soil, metal, ceramics, and/or plant products.

a. DNA Molecules

DNA molecules that can be included in the compositions include any DNA molecule that is of personal significance to the end user. For example, the DNA can be from a human, non-human animal or plant source. The DNA is preferably included in the personalized capsules in the form of microspheres. (FIGS. 1B and 2B). Typically the DNA is isolated from the source.

Preferably, the DNA does not include a vector. Optionally, the DNA molecules include one or more identification characteristics. The one or more identification characteristics includes unique information which can be used to verify that the personalizing substance was obtained from a particular source, e.g., human, non-human animal, or plant.

In a particular embodiment, the DNA is from a human.

No two people have the exact same sequence of DNA in their cells. The differences in the DNA in individual humans gives rise to the unique DNA profiles that can be used to distinguish individuals. In addition, the unique DNA profile of each individual provides a means for verifying that the personalizing substance is from a particular individual. Accordingly, incorporation of DNA into a carrier or into a tattoo ink provides a unique characteristic to the tattoo ink or carrier that may be verified, for example, through DNA sequencing or analysis of genetic markers.

The DNA may be coding or non-coding genomic DNA, coding or non-coding mitochondrial DNA or complementary DNA (cDNA). cDNA is synthesized from RNA using reverse transcriptase. The genomic DNA, mitochondrial DNA, and RNA for synthesis of cDNA may be isolated from any organism, including but not limited to humans, animals, and plants. In some embodiments, the DNA is isolated from a single organism, for example, a human. In other embodiments, the DNA is isolated from two or more organisms, for example, two or more humans.

In some embodiments, the DNA contained in the personalizing substance may be generated by PCR. For example, DNA comprising STRs may be amplified by PCR using primers that amplify three to five tetranucleotide repeat segments of the genomic DNA sample, optionally incorporating a detectable label, such as a radioactive or fluorescent label, as is known in the art. PCR primers for amplifying the DNA may be obtained from a commercial source or may be synthesized using methods known in the art. Software for design of PCR primers is well known in the art.

Examples of preferred STRs that may be amplified by PCR are set forth in Table 1 below. The skilled artisan will appreciate that additional suitable tetranucleotide and pentanucleotide repeats may also be amplified. One of the preferred qualities of suitable tetranucleotide DNA repeats is high heterozygosity (variability between individuals) in the subject population. Another preferred quality of suitable tetranucleotide DNA repeats is that they do not encode a biologically active product, for example, a protein, tRNA, rRNA, miRNA, or siRNA. A further preferred quality of suitable tetranucleotide DNA repeats is that they do not induce an immune response and produce no therapeutic action in the recipient.

TABLE 1
Preferred Repeats in DNA for amplification

| Human | Allele Distribution | Number of |
|---|---|---|
| Marker | (bp) | Repeats |
| D3S1358 | 98 to 146 | 8 to 20 |
| D5S818 | 133 to 169 | 7 to 16 |
| D7S820 | 215 to 247 | 6 to 14 |
| D8S1179 | 163 to 213 | 7 to 19 |
| D13S317 | 161 to 205 | 5 to 16 |
| D16S539 | 133 to 173 | 5 to 15 |
| D21S11 | 201 to 257 | 24 to 38 |
| D8S1106 | 109 to 133 | 7 to 13 |
| D1S518 | 182 to 198 | 13 to 17 |
| D6S1017 | 354 to 374 | 10 to 15 |
| D17S1304 | 197 to 213 | 10 to 14 |
| D4S2408 | 336 to 360 | 13 to 19 |
| D5S1467 | 173 to 189 | 8 to 12 |
| D19S245 | 225 to 249 | 16 to 22 |

The resulting PCR products are typically analyzed, for example, by electrophoresis, for the successful generation of tetranucleotide repeats and to confirm that the sample shows a relatively unique representation of a DNA sample from an individual.

i. Verification of Amplified DNA

In some embodiments, the DNA is analyzed to confirm that the DNA contained in the personalizing substance was obtained or generated from the desired source organism. For example, for DNA comprising STRs, the pattern of PCR products in the DNA contained in the personalizing substance may be compared to a control sample obtained from the source organism. The DNA contained in the personalizing substance may also be analyzed by DNA sequencing, for example cDNA sequencing or whole genome sequencing, to confirm that the DNA contained in the personalizing substance is from the desired source organism.

The sequencing of the DNA may be performed using methods known in the art. These include, but are not limited to basic sequencing methods, such as Sanger's method, Maxam-Gilbert sequencing and chain termination methods (França et al., Quarterly Review of Biophysics, 35(2):169-200, 2002), advanced methods and de novo sequencing, such as shotgun sequencing and bridge PCR (Braslavky et al., Proc. Natl. Acad. Sci, 100(7):3960-3964, 2003), or next-generation methods. Next-generation sequencing applies to genome sequencing, genome resequencing, transcriptome profiling (RNA-Seq), DNA-protein interactions (ChIP-sequencing), and epigenome characterization (de Magalhães et al., Ageing Res Rev. 9(3)315-323, 2010; Liu et al., Journal of Biomedicine and Biotechnology, 2012:1-11, article ID 251364, 2012; and Hall, The Journal of Experimental Biology, 209:1518-1525, 2007). Resequencing is necessary, because the genome of a single individual of a species will not indicate all of the genome variations among other individuals of the same species.

Next Generation sequencing encompasses a number of methods, including, but not limited to single-molecule real-time sequencing, massively parallel signature sequencing, (MPSS), Polony sequencing, 454 pyrosequencing, ion torrent semiconductor sequencing, DNA nanoball sequencing, heliscope single molecule sequencing, sequencing by ligation (SOLiD sequencing) and single molecule real time sequencing (SMRT). These methods are detailed and compared in Liu et al., Journal of Biomedicine and Biotechnology, 2012:1-11, article ID 251364, 2012, and Hall, The Journal of Experimental Biology, 209:1518-1525, 2007.

In some embodiments, the DNA contained in the personalizing particle is analyzed before the personalizing particle is combined with a carrier or in tattoo ink. In other embodiments, the DNA contained in the personalizing particle is analyzed after the personalizing particle is combined with a carrier or tattoo ink.

The DNA may be purified to obtain pharmaceutical/biologics grade DNA suitably free of contaminants.

ii. Percent Loading of DNA

Typically, percent loading for DNA in the microparticles ranges from 0.000001% to 0.1% weight of DNA to the total weight of the microparticles (% w/w). In preferred embodiments, the amount of DNA in the microparticles is less than 0.01% (w/w) DNA, more preferably the amount of DNA in the microparticles ranges from 0.001% to 0.00001% (w/w). These loading ranges are generally applicable to single-walled microparticles.

However, for embodiments, in which the microparticles are in metal particles or double walled microparticles, higher loadings of DNA may be used. It is expected that the structure of the double-walled microparticles protects the DNA from leaching out of the microparticles. In these embodiments, the amount of DNA in the microparticles may range from 0.000001% to about 5% weight of DNA to the total weight of the microparticles (% w/w), optionally from about 1%-5% (w/w).

For centimeter-sized particles, such as capsules, higher percent loadings of DNA may be used.

b. Sand

Exemplary sources of personalizing substances are provided in Table 2.

TABLE 2
Exemplary Personalizing Substances

| Source for | |
|---|---|
| Personalizing | |
| Substance | Personalizing Substance |
| White Beach Sand | Quartz (SiO₂) particles of different |
| | diameter ranges and limestone from coral |
| | or shells. |
| Dark Sand | Quartz (SiO₂) particles of different |
| | diameter ranges and magnetite. |
| Green Sand | Quartz (SiO₂) particles of different |
| | diameter ranges and chlorite |
| Rock | Quartz (SiO₂) particles of different |
| | diameter ranges and other trace elements |
| | that vary with geographical location. |

Sand consists predominately of silica (SiO₂) and other organic and inorganic minerals, such as calcium silicate (Ca₂SiO₄), calcium nitride (Ca₃N₂), silicon nitride (Si₃N₄), aluminum nitride (AlN₃), alumina (Al₂O₃), borazone “boron nitride” (BN), magnesium oxide (MgO), silicon oxysulfide (SiOS), lithium silicate (Li₂SiO₄), as well as other metal oxides/nitrides, as shown in Table 2.

c. Optional Identification Characteristics

Optionally, the personalizing substance may include one or more identification characteristics, which are unique to the source of the personalizing material, and which can be used to identify the source of the personalizing material. Exemplary personal identification characteristics for DNA include, but are not limited to, microsatellite markers such as short tandem repeats (STRs) and Simple Sequence Repeat (SSR) markers, single nucleotide polymorphisms (SNPs), and epigenetic markers, such as methylated DNA patterns. Any DNA sequence that is unique to the source organism may be used as a personal identification characteristic. A DNA sequence unique to a source organism may be identified by sequencing the entire sequence of the DNA isolated from the source organism, or a portion thereof, using sequencing methods known in the art such as Sanger sequencing or next generation sequencing, e.g. Illumina sequencing. DNA sequencing methods are well known in the art and are described, for example, in Sambrook, et al., Molecular Cloning. (4th ed.). Cold Spring Harbor, N.Y.: Cold Spring Harbor Laboratory.

i. Polymorphic Genetic Markers

DNA generally includes one or more polymorphic genetic markers.

Polymorphic genetic markers are highly variable regions of the genome which have contributed to the development of a variety of applications such as forensic DNA analysis and paternity testing that are used to unambiguously identify individuals.

Many polymorphic genetic markers have been identified over the last thirty years. For example, polymorphic genetic markers known as variable number of tandem repeats (VNTRs) are abundant and highly polymorphic regions of DNA containing nearly identical sequences, 14 to 80 bases in length, repeated in tandem (Jeffreys et al., Nature, 314:67-73 (1985); Wyman et al., PNAS, 77:6754-6758 (1980); and Nakamura et al., Science, 235:1616-1622 (1987)). The variation in these markers between individuals makes them useful for identifying particular individuals. VNTRs may be detected from small amounts of DNA using polymerase chain reaction (PCR) (Kasai et al., Journal of Forensic Sciences, 35(5):1196-1200 (1990)). Size differences in the amplified PCR products are detected on agarose or polyacrylamide gels. However, the finite number of VNTRs limits the widespread applicability of this method, which in turn led to the identification of short tandem repeats (STR).

ii. Short Tandem Repeats (STR)

The personalizing substance may contain a human DNA sequence such as dinucleotide STR, a trinucleotide STR, a tetranucleotide STR and a pentanucleotide STR.

STRs can contain tandem repeat sequences that differ by two (dinucleotide), three (trinucleotide), four (tetranucleotide) or five (pentanucleotide) base pairs. It is estimated that there are approximately 50,000 to 100,000 dinucleotide repeats in the human genome. Trinucleotide and tetranucleotide repeats are less common; the human genome is estimated to contain 10,000 of each (Tautz et al, Nuc. Acids Res., 17:6464-6471 (1989); and Hamada et al., PNAS, 79:6465-6469 (1982)). The use of tetranucleotide and pentanucleotide STRs allows better discrimination of differences between individual subjects relative to the shorter sequences (Weber et al., Am J Hum Genet, 44:388-396 (1989).

STRs can be amplified by a polymerase chain reaction, and are highly abundant and polymorphic (variable from individual to individual). Tetranucleotide repeats are a preferred personal identification molecule for use as a personalizing substance because the size of PCR products from human tetranucleotide repeat regions typically varies between individuals. For example,

PCR products of two different sizes are observed based on the inheritance for each individual of one copy of the polymorphic marker from each parent. Each inherited copy contains a variable number of tetranucleotide repeats. Thus, two unrelated individuals likely will produce different sized PCR products from the same tetranucleotide polymorphic marker. As a greater number of different tetranucleotide repeat regions are compared between individuals, the probability of those individuals sharing the identical pattern of PCR products decreases.

iii. Single Nucleotide Polymorphisms (SNPs)

A single nucleotide polymorphism is a DNA sequence variation occurring commonly within a population (e.g. 1%) in which a single nucleotide —A, T, C or G—in the genome (or other shared sequence) differs between members of a biological species or paired chromosomes. For example, two sequenced DNA fragments from different individuals, AAGCCTA to AAGCTTA, contain a difference in a single nucleotide. SNPs may fall within coding sequences of genes, non-coding regions of genes, or in the intergenic regions (regions between genes). SNPs within a coding sequence do not necessarily change the amino acid sequence of the protein that is produced, due to degeneracy of the genetic code. SNPs in the coding region are of two types, synonymous and nonsynonymous SNPs. Synonymous SNPs do not affect the protein sequence while nonsynonymous SNPs change the amino acid sequence of protein. The nonsynonymous SNPs are of two types: missense and nonsense. SNPs that are not in protein-coding regions may still affect gene splicing, transcription factor binding, messenger RNA degradation, or the sequence of non-coding RNA. Gene expression affected by this type of SNP is referred to as an eSNP (expression SNP) and may be upstream or downstream from the gene. SNPs without an observable impact on the phenotype (so called silent mutations) are still useful as genetic markers in genome-wide association studies, because of their quantity and the stable inheritance over generations.

4. Biocompatible Materials

The capsules may be formed from one or more biocompatible materials. The materials are preferably corrosion resistant. Suitable materials include implantable grade synthetic plastics or metals like stainless steel, cobalt-chromium, tantalum, titanium or the alloys of these metals. Other useful materials include, but are not limited to ceramics, zirconium and zirconium alloys and oxidized zirconium.

Implantable grade synthetic plastics include PEEK (polyether ether ketone), PEKK (polyether ketone ketone), polyethylene, ultra high molecular weight polyethylene, polyphenylsulfone, polysulfone, polythermide, acetal copolymer, polyester woven or solid or implantable grade lennite UHME-PE.

Cobalt-chromium alloys are hard, tough, corrosion resistant, bio-compatible metals.

Pure titanium is generally used in implants where high strength is not necessary, and accordingly, can be used to make the nano/microparticles or the centimeter sized capsules disclosed herein.

Titanium alloys are bio-compatible in nature. They commonly contain amounts of vanadium and aluminum in addition to titanium. The most used titanium alloy in knee implants is Ti6Al4V. Titanium and titanium alloys have great corrosion resistance, making them inert biomaterial.

Tantalum is a type of pure metal, which has excellent biological and physical properties, namely flexibility, corrosion resistant and biocompatibility.

Zirconium displays excellent corrosion resistance in many aqueous and non-aqueous media and for this reason has seen an increased use in the chemical process industry and in medical applications. Oxidized zirconium and Zirconium alloys are disclosed for example in U.S. Pat. No. 7,473,278.

In a preferred embodiment, the metal is titanium.

B. Pharmaceutically Acceptable Carriers

In certain embodiments, the compositions described herein are formulated for injection into the skin of a human. For example, the composition may include a suitable biocompatible carrier for delivery to a human via injection. Suitable carriers include any alcohol, including but not limited to ethyl alcohol, isopropyl alcohol, or water. Suitable carriers also include any combination of alcohol and water. Typically the amount of alcohol in the carrier ranges from about 5% to about 30% (w/w), and the amount of water in the carrier ranges from about 40% to about 70% (w/w). In preferred embodiments, the carrier is a solution of 60% water, 30% glycerin (glycerol), and 10% ethanol. Other carrier solutions including 55% water, 30% glycerin and15% ethanol; 50% water, 30% glycerin, and 20% ethanol; 45% water, 30% glycerin, and 25% ethanol; or 40% water, 30% glycerin and 30% ethanol, are also contemplated.

### III. Methods Of Making

The particles disclosed herein can be made using conventional methods of making nano- and microparticles or using three dimensional (3D) printing.

Allen et al., disclose methods to assemble DNA functionalized microparticles into a colloidal gel, and to extrude this gel with a 3D printer at centimeter size scales. ACS Biomater. Sci. Eng., 1:19-26 (2015); See also, Moon, et al., Tissue Eng Part C., 15:1-9 (2009). The particle, for example, a capsule with a predetermined cavity is produced by 3D printing, following which the cavity is filled with the personalizing substance and then sealed. In some embodiments, the capsule has a cap, which can be snapped on, screwed in, or screwed on to close the cavity and lock the cap in place. In these embodiments, one end of the capsule is configured to receive the cap. Alternatively, the capsule is initially manufactured as two halves, with one end of each half configured to receive an end of the other half, as described above. Method for making metal drug delivery devices, such as brachytherapy seeds can be adapted for making the capsules disclosed herein. For example, U.S. Pat. No. 6,163,947 (and the references cited therein) discloses a method of making a hollow-tube brachytherapy device.

Other methods for making the particles disclosed herein are known in the art, and include, but are not limited to soldering, laser soldering, laser welding, manual closure—screw cap, sintering, including direct metal laser sintering (DMLS), metal injection molding, amalgamation, friction stir welding, cold spray process, electrolytic deposition, cold isostatic pressing, uni axial pressing, pressure welding/inertial welding, hot isostatic pressing, co-extrusion/drawing, hammer forging, vacuum impregnation, gas atomization, vapor deposition, static casting, centrifugal casting, pressure casting and weld overlay.

A. DNA Isolation and Amplification

Methods of isolating genomic DNA, mitochondrial DNA and RNA, and methods of cDNA synthesis are well known in the art and are described, for example, in Sambrook, et al., Molecular Cloning. (4th ed). Cold Spring Harbor, N.Y.: Cold Spring Harbor Laboratory.

In some embodiments, the DNA contained in the personalized particle is isolated directly from an organism, such as genomic DNA or mitochondrial DNA. In other embodiments, the DNA contained in the personalized particle is amplified from a sample collected from the organism, for example by polymerase chain reaction (PCR). Multiple DNA segments for tetranucleotide PCR amplification typically may be amplified in a single tube. Such multiple amplification of several DNA regions is known in the art as multiplex PCR. The multiple PCR products are separated as known in the art, for example, by electrophoresis, and an instrument reads the electrophoresis gel or image to automatically analyze the sizes of the PCR products. In some embodiments, the DNA contained in the personalized particle is cDNA reverse transcribed from RNA isolated from the organism. The DNA may be sequenced so that verification steps described below may be performed. (Sambrook, et al., Molecular Cloning. (4th ed.). Cold Spring Harbor, N.Y.: Cold Spring Harbor Laboratory).

Preparation of DNA samples for use as a personalizing substance may proceed as follows, although other methods of preparing analogous DNA samples are known to the skilled artisan. One preferred method includes the following general steps:

A sample for preparation of the DNA contained in the personalized particle is collected from a sample of cheek swab, skin, hair, saliva, or blood or other tissue from an organism as is known in the art. A cheek swab sample is preferred. Protocols for collecting and handling the sample are known in the art.

For example, a DNA isolation kit suitable for isolating genomic DNA from buccal cells, may be used to isolate DNA from the cheek swab. These kits are commercially available and usually generate 0.5-2 micrograms of total DNA. Desirable genomic regions containing polymorphic genetic markers (such as STRs and SNPs) of the isolated DNA are then amplified via PCR to generate micrograms, typically from 1 to 10 micrograms, of DNA to be used as a personalizing substance. The amplified DNA may be sequenced so that verification steps described below may be performed. This amplified DNA is the personalizing substance that is encapsulated into particles.

B. Isolation of Other Personalizing Substances

The particles disclosed herein may contain may contain silicon dioxide particles extracted from a soil or rock sample. Suitable extraction techniques are known. Following extraction, the particles may be ground by conventional means to reduce their size to less than 1 micron, optionally the particles are then screened to obtain a population of particles having a size range for encapsulation, or micronized to produce nanoparticles of suitable size, typically from about 1 to about 1000 nm in diameter.

In some embodiments, the personalizing substance comprises particles of a metal or ceramic object having meaning to a person receiving the substance. For example, such metal or ceramic objects can be ground, screened and extracted to remove unwanted components.

C. In Vitro Assay for Verification of Non-Release

Following delivery to an individual's skin, the encapsulated material remains in the particles, and the particles do not erode. The encapsulated material is not released from the particles. A simple in vitro test can be used to confirm that the particles will not release the encapsulated material following delivery to an individual's skin. For example, after formation of particles containing an encapsulated personalizing substance, the particles can be immersed in aqueous solution or buffer at pH 7.4 and temperature of 37° C. in a bath for at least about 1 month. Samples are removed periodically, such as after 1 hour, after 1 day, after 1 week, and after 1 month and analyzed using a suitable detection method to determine if any traces of the encapsulated material are in the aqueous solution, buffer, or supernatant.

Suitable detection methods are known in the art. For example, suitable methods for detection of whether any DNA is released include detection of the fluorescence of labeled DNA released into the aqueous solution, buffer, or supernatant and/or PCR amplification of the aqueous solution, buffer, or supernatant. PCR methods for detecting low levels of DNA in a sample are known in the art. See, for example, Sambrook, et al., Molecular Cloning. (4th ed.). Cold Spring Harbor, N.Y.: Cold Spring Harbor Laboratory. Conventional PCR and real-time PCR with real-time monitoring of amplification may be used to detect any DNA release. The PCR amplification may use the same primers and amplification conditions as those used for amplification of DNA prior to encapsulation. The PCR amplification may follow up to 50 amplification cycles and generates a detectable number of amplified DNA molecules, if any DNA is present in the aqueous solution, buffer, or supernatant, referred to as “the amplified product”). Following PCR, the amplified product, if present, may be detected by conventional gel electrophoresis techniques or UV-Vis spectrometry for detecting double-stranded DNA. See, for example, Sambrook, et al., Molecular Cloning. (4th ed.). Cold Spring Harbor, N.Y.: Cold Spring Harbor Laboratory. Presence of an amplified product following the process described above indicates release of DNA from the microparticles, and absence of amplified product indicates that DNA was not released from the microparticles.

Alternatively, or optionally, the personalizing DNA may be partially or fully labeled with fluorophores, such as Alexa Fluor® dyes (Molecular Probes, Inc.). The labeled DNA may be used to confirm that the DNA was successfully encapsulated, such as with flow cytometry of the encapsulated particles. Alternatively or additionally, the labeled DNA may be used to determine whether any of the encapsulated DNA will be released following delivery to an individual's skin. This test may be performed by measuring the fluorescence of the aqueous solution, buffer, or supernatant in which empty microparticles or those encapsulating labeled DNA were tested for DNA release in an in vitro method, such as described above.

For non-DNA personalizing substances, suitable detection methods include, IR, mass spectrometry, for example, isotope-ratio mass spectrometry (IRMS) or liquid chromatography mass spectrometry (LC-MS).

### IV. Method of Using

The compositions described herein may be used on their own, or in combination with a carrier for delivery to the skin, typically via injection. In some embodiments, the composition is administered the skin in combination with tattoo ink, using a suitable tattoo needle. In other embodiments including larger particles such as centimeter sized particles, the particles can be implanted into a desired site using known devices for injecting particles of similar sizes to a desired location, for example, using devices for implanting microchips or brachytherapy seeds. The particles can also be implanted using microdermal/transdermal implantation techniques.

The use of the composition may be chosen by the end user. For example, the compositions may be used by the end user to preserve a substance of personal significance for a long period of time. The end user may store the composition, or chose to present the composition as a gift to another individual. Alternatively, the end user may choose to use the composition as an additive to other substance.

In some embodiments, the compositions are administered via injection at a desired skin site of an individual. Typically, the site contains a marking or a marking is added to the site, such as a small tattoo, to indicate the presence of the encapsulated personalizing substance in that location.

Verification of Personalizing Substance Origin

A verification step may be made prior to or subsequent to encapsulation, optionally verification may occur after the personalizing substance is placed in the skin of an individual.

Exemplary identification characteristics for DNA include, but are not limited to, microsatellite markers such as short tandem repeats (STRs) and Simple Sequence Repeat (SSR) markers, single nucleotide polymorphisms (SNPs), and epigenetic markers, such as methylated DNA patterns. Any DNA sequence that is unique to the source organism may be used as a personal identification characteristic.

### V. Kits

Kits for obtaining a personalizing substance from an end user and kits for delivering the encapsulated personalizing substance to the end user are provided.

The kits include equipment for obtaining a sample of the personalizing substance. The equipment may be tailored to the nature of the personalizing substance that will be provided. For example, if the personalizing substance is DNA, then the kit may include a foam or cotton-tipped cheek swab, a protective container for the swab, and instructions for use. If the personalizing substance is sand, then the kit may include a waterproof container and instructions for use. The end user uses the cheek swab for example, to obtain a sample from a human or non-human animal of interest. Then the end user mails or otherwise delivers the sample to a lab. The lab isolates, amplifies (if needed), and purifies (if needed) the DNA, and then encapsulates the DNA in particles as described herein. In some embodiments the encapsulated DNA is lyophilized into a powder. Optionally, the powder is added to a carrier suitable for injection. The particles, powder, solution can delivered to the customer.

In other embodiments, the kits provide the final product for use by the end user. In these embodiments, the kits may include a personalized substance, a carrier, and instructions for use. The kits may be customized to the preference of the end user. For example, the kits contain the personalizing particles and a carrier. In other embodiments, the kits may contain the personalizing particles and a suitable needle for injection.

We claim:

1. A composition comprising a personalizing substance wherein the personalizing substance is encapsulated in a biocompatible particle/capsule, and wherein the particle does not release the personalizing substance.

2. The composition of claim 1, wherein the particle size is between 0.1 cm and 3 cm in diameter and/or height, preferably ranges from 0.1 to10 mm or from 10 mm to 3 cm.

3. The composition of claim 1, wherein the capsule comprises a cavity ranging in diameter from 0.5 mm to 10 mm, and wherein the cavity comprises the personalizing substance.

4. The composition of claim 1, wherein the personalizing substance is selected from the group consisting of DNA, sand, soil, metal, cremated ash, ceramics, and plant tissue.

5. The composition of claim 4, wherein the personalizing substance is isolated DNA, and wherein the DNA further comprises a personal identification characteristic selected from the group consisting of short tandem repeats (STRs), single nucleotide polymorphisms (SNPs), epigenetic markers, and methylated DNA patterns.

6. The composition of claim 2, wherein the capsule comprises a first half and a second half, wherein one end of the first half is configured to receive one end of the second half.

7. The composition of claim 1, wherein the particle is formed from an implantable grade synthetic plastic or a metal.

8. The composition of claim 1, wherein the particle is formed from a metal selected from the group consisting of stainless steel, cobalt-chromium, tantalum, zirconium, oxidized zirconium, titanium and alloys of these metals.

9. The composition of claim 1, wherein the personalizing substance is isolated DNA, and wherein the particles comprise up to 0.01% (w/w) DNA.

10. The composition of claim 1, wherein the personalizing substance is not DNA, and wherein the particles comprise up to 10% (w/w) of the personalizing substance.

11. The composition of claim 1, further comprising a pharmaceutically acceptable carrier.

12. The composition of claim 2, wherein the DNA is synthetic DNA or isolated genomic DNA

13. A method of encapsulating a personalizing substance for administering to the skin of an individual, comprising:
(a) isolating the personalizing substance from a source organism or source material; and
(b) encapsulating the personalizing substance in a particle comprising an implantable grade synthetic plastic or a metal selected from the group consisting of stainless steel, cobalt-chromium, tantalum, zirconium, oxidized zirconium, titanium and alloys of these metals.

14. The method of claim 13, wherein the personalizing substance is isolated genomic DNA or synthetic DNA.

15. The method of claim 14, further comprising amplifying the isolated DNA prior to step (b).

16. The method of claim 13, wherein the isolated DNA is human DNA.

17. The method of claim 13, further comprising:
(c) analyzing the personalizing substance isolated from the source organism or source material;
(d) analyzing the personalizing substance encapsulated in the particle; and
(e) comparing data obtained in step (d) to data obtained in step (c) to confirm that the personalizing substance isolated from the source organism or source material is the same as the personalizing substance encapsulated in the particle.

18. A method of delivering a personalizing substance to the skin of a recipient comprising
injecting into the recipient's skin the composition of claim 1.

19. The method of claim 18, wherein the personalizing substance is selected from the group consisting of isolated DNA, sand, soil, metal, cremated ash, ceramics, and plant tissue.

20. The method of claim 18, wherein the particles have a size ranging from 1 micron to 5 microns.

21. The method of claim 19, wherein the personalizing substance is isolated DNA and wherein the isolated DNA further comprises a personal identification characteristic selected from the group consisting of short tandem repeats (STRs), single nucleotide polymorphisms (SNPs), epigenetic markers, and methylated DNA patterns.
